(19) United States
(12) Patent Application Publication
(10) Pub. No.: US 2003/0114366 A1
(43) Pub. Date: Jun. 19, 2003

(54) MICROFABRICATED PARTICLES AND METHOD FOR TREATING SOLID TUMORS

(76) Inventors: FRANCIS J. MARTIN, SAN FRANCISCO, CA (US); MAURO FERRARI, DUBLIN, OH (US)

Correspondence Address: FISH & RICHARDSON P.C., 500 ARGUELLO STREET, SUITE 500, REDWOOD CITY, CA, 94063, US

(21) Appl. No.: 09/479,390

(22) Filed: Jan. 6, 2000

Related U.S. Application Data
(60) Provisional application No. 60/115,426, filed on Jan. 11, 1999.

(51) Int. Cl. A61K 38/17; A61K 9/14; A61K 9/50

(52) U.S. Cl. 514/12; 424/499

(57) ABSTRACT

Microfabricated, asymmetrical, reservoir-containing particles for use in the intravenous delivery of cytotoxic agents such as melittin to tumors is disclosed. The particles have a selected shape and uniform dimensions preferably in the 1 μm to 10 μm range. The reservoirs open to the face of the particle and are filled with a solution or suspension of the therapeutic agent and selected excipients. The drug/excipient solution may be dried by standard techniques. The excipients are selected to delay the dissolution/release of the agent from the particle reservoirs for 1-48 hours after the particle suspension is rehydrated and injected. Alternatively, the pore is plugged with an erodable material or covered with a semipermeable membrane. The face of the particle is grafted with a layer of specific ligands designed to quickly bind the particle to the surface of either tumor cells or the vascular endothelial cells, which form tumor capillaries. The cytolytic agent, which is released from the reservoirs after binding, is presented directly to the surface membranes of target cells. The locally high concentration of cytolytic agent achieved in the circumscribed volume between the face of the particle and the juxtaposed cell provides for efficient entry of the cytolytic agent directly into the surface membrane of the target cell leading to cell lysis ans death. Also disclosed are microfabrication methods for making such particles and a method of treating cancer patients with such particles.

### FIELD OF THE INVENTION

The present invention relates to microfabricated devices, and more particularly to microstructural particles for use in delivering cytotoxic drugs to tumors.

### BACKGROUND AND SUMMARY OF THE INVENTION

The present invention includes asymmetric microparticles for intravenous administration in treating tumors. The particles are characterized by(I) uniform sizes, e.g., in the range 0.5 to 10 μm, (ii) at least one internal reservoir which communicates through at least one pore with the front face of said particle, (iii) where each reservoir contains a releasable cytotoxic agent, (iv) the pore and/or reservoir filled or covered with release-delaying material, and (v) a layer of ligand molecules chemically grafted to the same face of the particle as the pore openings.

In various embodiments:

(1) the release-delaying material delays release of the cytotoxic agent for 1-48 hours after injection;

(2) the reservoir or pore is covered with a semipermeable membrane;

(3) a coating of a hydrophilic polymer, such as polyethylene glycol, effective to extend the circulation lifetime of the particles in the bloodstream, is chemically grafted to all faces of the particle surface, and the ligand may be coupled to a spacer arm sufficient to extend the ligand beyond the hydrophilic polymer layer.

(4) following release from the reservoir, the cytotoxic agent is a cytolytic agent that enters the surface membrane of juxtaposed cells and causes cytolysis, where the cytolytic agent may be bee venom melittin, paradaxin, hemolysin, amoebapore, pilosulin, magainin, lentivirus lytic peptide, NK-lysin or perforin.

(5) the particle's shape is disc-like or hexagonal-like;

(6) the particles' fron face is grafted with a layer of reactive amino or thiol groups by plasma (glow) discharge or by sialylation methods, where the layer of reactive amino or thiol groups is used to chemically link ligands to the front face of the particle;

(7) the ligand binds to receptors overexpressed on tumor cells or angiogenic vascular endothelial cells, where the ligand may be FGFb, VEGF, c-erbB-2 ligand, RGD-type tumor targeting cyclic peptides or folate;

(8) the ligand is an antibody or antibody fragment which binds to receptors overexpressed on tumor cells or angiogenic vascular endothelial cells, where the antibody may bind to growth factor receptors overexpressed on tumor cells or angiogenic vascular endothelial cells, where the growth factor may be FGFr, VEGFr or c-erbB-2 receptor, or the antibody may bind to integrin receptors overexpressed on tumor cells or angiogenic vascular endothelial cells, where the integrin receptor may be e-selectin, p-selectin or v3;

(9) the particles are formed of a biodegradable polymer material, and/or contain a radioactive material;

(10) the release-delaying material is co-mixed with the cytotoxic agent held within the reservoir, or is layered above the cytotoxic agent within the reservoir, or forms a plug within or covering said pore.

(11) the release-delaying material is a semipermeable membrane covering the pore; and

(12) the release-delaying material consists of gelatin, polyethylene glycol, fatty acids or esters, polyvinyl pyrrolidone, starch, dextrans or maltodextrins, hydrocolloidal gums or mucilages, waxes, polyacrylic acids, shellac, cellulose acetate phthalate or carboxymethylcellulose.

In another aspect, the invention includes a microfabrication method for producing asymmetrical particles of the type described above. The method includes exposing a sheet of particle-forming material to a photoablating light source through a series of photomasks forming a reticular lattice pattern on the sheet corresponding to the desired particle external size, shape and interior volume and continuing the exposure until the desired particles are formed. In another aspect, the invention includes a method for treating patients with solid tumors, by administering particles of the type described above by intravenous injection.

These and other objects and features of the invention will become more fully apparent when the following detailed description is read in conjunction with the accompanying drawings.

### BRIEF DESCRIPTION OF THE DRAWINGS

FIG. 1 depicts the structural features of a typical microparticle of the present invention. Each particle, 100, contains at least one reservoir filled with a cytotoxic drug, 102, and at least one pore or channel connecting the reservoir with the front face of the particle, 104. In the example illustrated, the pore is filled with a erodible material, 106, which serves to delay the release of the drug for 1-48 hours after rehydration and injection. The front face of the particle is grafted with a layer of specific ligands, 102, eg. FGF, which serve to bind the microparticle to receptors expressed on tumor cells or angiogenic blood vessels.

FIG. 2 illustrates drug-filled particles binding via ligands chemically grafted the particle face to receptors over-expressed on the endothelial cells, which form newly sprouted blood vessels in tumors. Each internal reservoir of the particle contains a dry mixture of the cytolytic drug, eg. melittin. The pore connecting the reservoir with the front face of the particle is plugged with an erodible material. As the plug erodes, the dry agent is hydrated and solvated by the influx of water and moves outwardly, by diffusion entering the juxtaposed surface membrane of the target cell (B). Entry of the cytolytic agent causes colloid osmotic lysis of the cell and cell death (C).

FIGS. 3A-3E illustrate typical micro-particles of the present invention. Each is made of a substrate material (300) and contains blind reservoirs such as 302 in FIGS. 3A-3D and 304 in FIG. 3E. Possible shapes include disc-like (FIG. 3A), cup-like (FIG. 3B), hexagonal (FIG. 3C) and ring-like (FIG. 3D). The typical diameters of such particle (D in FIG. 3D) range from 1-10 μm.

FIGS. 4A-4B illustrate the structural features of micro-particles made. Each particle (402) contains uniform, cylindrical, blind pores (404). A layer of reactive chemical groups such as primary amino groups may be introduced onto the face of the particles and specific ligands (408) grafted via these groups to the particle face. A drug/excipient solution is filled into the pores (FIG. 4A) and dried (FIG. 4B).

FIG. 5 shows the sequence of step in the top-down fabrication of micro-particles using a combination of vapor or thin film deposition followed by photolithography.

FIG. 6 shows an example of one the reaction sequence that can be used to graft protein ligands, such as FGFb, onto the face of the particles. Surface amino groups are reacted with a heterobifunctional reagent such as SMCC to introduce thiol-reactive maleimide groups. Thiolated lectins such as wheat germ agglutinin or tomato lectin are then reacted with the thiol-reactive groups to create thiol-ether linkages between the maleimide and thiols on the proteins.

### DETAILED DESCRIPTION OF THE INVENTION

### I. Definitions

Unless indicated otherwise, the terms below have the following meaning.

“Particles” or “microparticles” or “microfabricated structures” or “microfabricated particles” are particles formed by microfabrication methods.

“Microdevices” or “microfabricated devices” are particles that have been additional prepared to include biological agents as coatings and/or therapeutic agents.

“Microfabrication methods” refer methods employing photomasking or patterned beam irradiation of a substrate to produce desired surface pattern features in the substrate. Exemplary microfabrication methods include photolithography, x-ray lithography and electron-beam lithography.

“Bioerodible” refers to a material that is dissolvable in physiological medium (e.g., an erodible metal), or a biocompatible polymeric material that can be degraded under physiological conditions by physiological enzymes and/or chemical conditions, e.g., conditions found in the GI tract.

A. Anti-angiogenesis as a Strategy for Treatment of Cancer

Primary tumors are generally not the principal cause of morbidity and mortality among cancer victims. Indeed, highly effective treatments exists for the majority of primary lesions, including surgery, radiation and focused chemotherapy. Metastatic disease presents a far greater treatment challenge as metastatic lesions typically form in multiple sites and deep in vital organs. Surgery is only partially effective and radiation exposure to multiple sites in the body can lead to unacceptable cumulative systemic toxicity, well before the disease process is under control. Systemic chemotherapy using cytotoxic or biological agents is the only treatment option for many patients with advanced metastatic cancer. Many tumors respond to initial courses of chemotherapy. Unfortunately, after exposure to multiple courses and drugs, cancer cells become resistant and fail to respond to further therapy. Multiple drug resistance (or MDR) is one of the most vexing problems faced by medical oncologists, leading to treatment failures in the vast majority of patients with metastatic disease.

Metastatic tumors develop when a small number of cells (or clumps of cells) detach from primary tumors, enter and move through blood vessels or lymphatics, invade tissues at distant anatomical sites and form metastatic foci. The in situ proliferation of such cells, and formation of secondary micrometastatic lesions, must rely on nutrients to be provided by normal blood vessels supplying the area. Once the tumors have grown to a few mm³ in volume, normal vessels are insufficient to support further tumor growth. Further growth of metastatic tumors is supported by factors, secreted by the tumor cells themselves, which, in a coordinated fashion, cause new blood vessels to sprout from existing ones. This process, known as angiogenesis, produces a network of blood vessels, which supply nutrients to the growing tumor mass and provide yet another avenue for spread of the disease.

During angiogenesis, tumor cells secrete growth factors such as vascular endothelial growth factor (VEGF) and basic fibroblast growth factor (FGFb), both of which stimulate the endothelial cells of normal blood vessels to proliferate and mobilize. The tumor cells also elaborate enzymes, metalloprotinases, which carve out tiny channels in the tissue matrix into which the endothelial cells migrate, eventually forming closed tubes (capillaries). Migration of endothelial cells is guided by the interaction of integrins expressed on such cells during proliferating with RGD-containing matrix proteins such as fibronectin. One strategy for preventing or treating metastatic disease, which is gaining favor among oncologists, is to intervene in the process of angiogenesis. By preventing new blood vessels from sprouting, or killing the endothelial cells, which form existing tumor capillaries, metastatic tumors may be prevented from growing and spreading. Moreover, no resistance would be expected to develop to such therapy. A blood supply is essential for tumor growth and without it tumors would regress.

B. Overview of the Invention

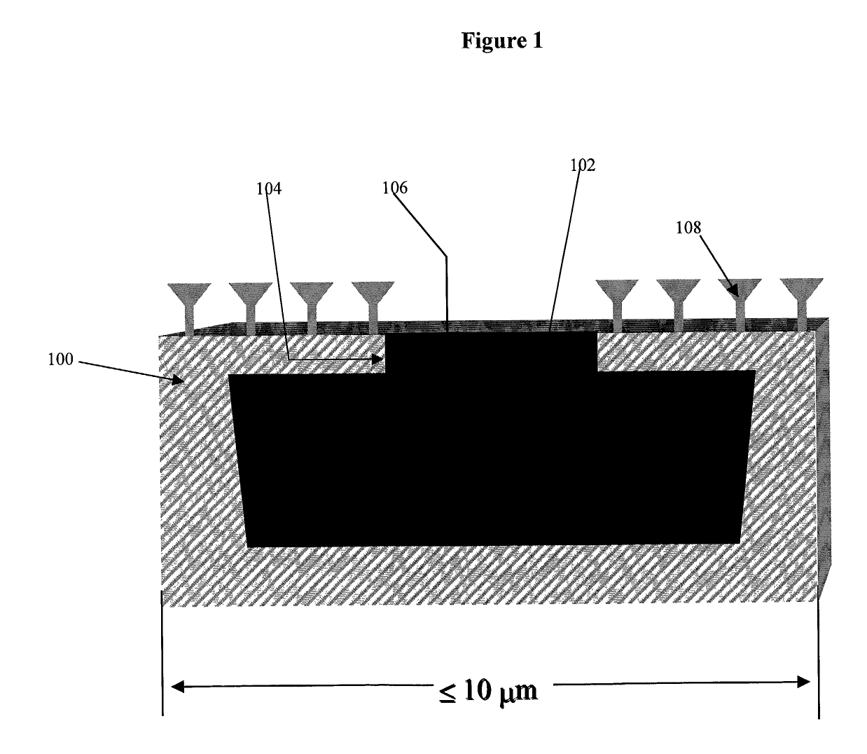

FIG. 1 shows a microparticle 100 formed in accordance with one embodiment of the invention. The microparticle has at least one drug reservoir 102 connected to the front face of the particle by pore 104 that is plugged with an erodible material 106. The face of the particle to which the pore opens is grafted with a layer of specific ligands 108. The plug material may be bioerodible, to deliver its drug load at a selected time, e.g., 1-48 hours, after parenteral administration, e.g., IV injection, or it may be covered, preferably on its inner side, with a bioerodible film that allows drug release through the membrane after the film bioerodes. The pore may alternatively be covered with a semipermeably membrane which allows water (but not solutes) to enter. In this case, dry particles are suspended in an aqueous vehicle (such as saline) and immediately injected. Immediately after resuspension, water begins to enter the reservoir through the semipermeable membrane, solvating the drug (which doses not pass through the membrane). As water continues to enter, osmotic pressure builds within the reservoir and the membrane burst, allowing release of the hydrated drug.

A variety of permeable materials suitable for microfabrication are contemplated, such as disclosed in U.S. Pat. No. 5,200,051 for “Wholly Microfabricated Biosensors and Process for the Manufacture and Use Thereof”, and U.S. Pat. No. 5,212,050 for “Method of Forming Perm-selective Layer”. Other exemplary biocompatible materials may be found, for example, in “Biomaterials Science”, Ratner, B., et al, eds., Academic Press (1996), and references cited therein. The particles may be coated with polyethylene glycol (PEG) chains, typically in the 2-10K molecular weight range, at a surface density effective to maintain the particles in blood circulation time with a half life of at least 2-12 hours. Methods for derivitizing a polymer substrate having surface amine, carboxyl, alcohol, or aldehyde groups, for example, are known.

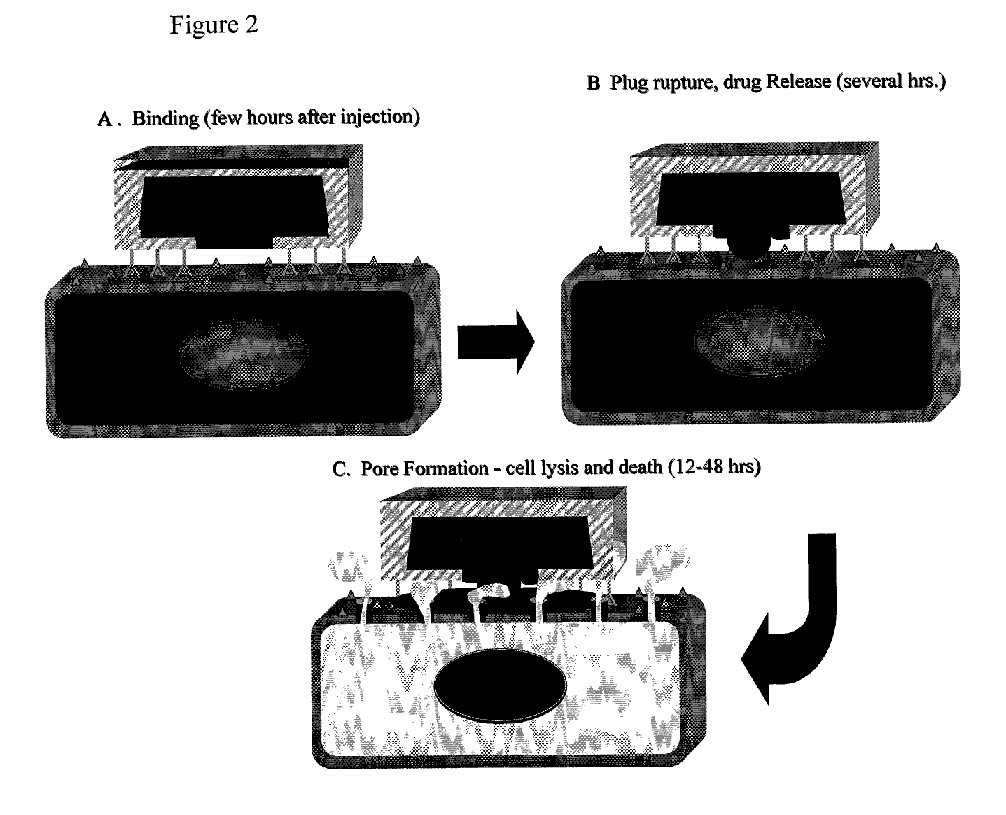

Carried on the interior of the microparticle is at least one drug reservoir 102 which carries a cytolytic agent, e.g., melittin. The microparticle includes any array of ligands molecules, grafted to the same face as the pore openings, which bind the particle to receptors overexpressed on tumors cells or the angiogenic vascular endothelium that supplies blood to tumors. In use, the particles are injected intravenously, where they circulate in the bloodstream and are carried to tumor sites. Here the particles bind to proliferating vascular endothelial cells in regions of tumors undergoing angiogenesis, as indicated at A in the FIG. 2. After erosion of the plug material or film covering the pore (shown at B), drug is released within the circumscribed volume between the face of the microparticle and the surface membrane of the cell. Entry of the cytolytic drug into the cell membrane produces cell lysis (shown at C), which then leads to cell death. Death of the vascular endothelial cells forming the blood vessels, which supply tumors, leads to tumor shrinkage and eradication.

Particles of the type described can be constructed according to known microfabrication methods, or alternatively, according to novel methods such as described in co-owned U.S. provisional patent application for “Asymmetric Drug-Delivery Microparticles”, and co-owned provisional application for “Microfabricating Biodegradable Devices,” both of which are attached hereto and incorporated herein by reference.

C. Unique Geometry Provided by Microfabrication

“Top-down” fabrication of micro-devices, using techniques perfected by the electronics industry, provides the means to create microscopic particles with a unique combination of structural features useful for the present invention. Such particles can be made with extremely precise sizes and shapes and can contain pores, which, for the purposes of the present invention, act as reservoirs enabling the particle to transport chemotherapeutic drugs. Moreover, the particles may be asymmetrical. For example, the pores or reservoirs can be made to open only to the top face of the particle. The top face (containing the pore openings) can also be chemically modified to contain reactive chemical groups such as primary amino or thiol groups, which can be used to chemically graft protein or other types of ligands to this face only. As will become evident in the discussions below, the unique geometry provided by such microfabrication methods is useful to create the particles of the present invention.

D. Microfabrication Schemes

The so-called “top-down” approach employs a combination of thin film deposition methods plus photolithography, photoablation and etching techniques to deposit and cells membrane. Entry of a few as 10⁶-10⁷ molecules of melittin will cause lysis and death of the target endothelial cell. Melittin molecules which do not enter the juxtaposed target cell membrane, and melittin molecules released from particle elsewhere in the body (i.e., those which have not bound to endothelial target cells), is inactivated by binding to albumin and thus does not cause toxicity to normal cells.

TABLE 1
Partial List of Lytic Peptides

| Name of | | |
|---|---|---|
| Lysin | Source | Comments |
| Melittin | Apis mellifera | 26 amino acid peptide: |
| | (honey bee) | GIGAVLKVLTTGLPALISWIKRKRQQ- |
| | | NH₂ |
| paradaxin | Pardachirus | Short peptide |
| | marmoratus | |
| | (red sea Moses | |
| | sole) | |
| hemolysin | Staphylococcus | Short peptide |
| | aureus | |
| Amoebapore | Entamoeba | 77 amino acid residues arranged in 4 |
| | histolytica | alpha-helical domains |
| pilosulin | Myrmecia | 56-amino acid residues polypeptide |
| | pilosula | |
| | (jumper ant) | |
| magainin | Xenopus laevis | Short peptide |
| | (skin) | |
| Lentivirus | HIV-1 | Carboxy-terminal 29 amino acid |
| lytic | | residues of transmembrane |
| peptide | | glycoprotein |
| NK-lysin | Cytotoxic T | |
| | lymphocytes | |
| perform | Natural Killer | N-terminal 22-residue domain |
| | lymphocytes | |
| | (NK) | |

E. Filling Reservoirs with Drug

Mixtures of drug plus excipients which provide defined dissolution rates. In one approach, prior to filling of the reservoirs, a solution of the melittin or similar cytolysin is mixed with a solution of a water-soluble excipient. The mixture is then dried within the reservoirs. The excipient is selected to delay rehydration and dissolution of the mixture for 1-48 hours after injection into the bloodstream. Suitable excipients are listed in Table 2.

TABLE 2
Excipients and Plug Materials for Use in
Delayed Release of Cytolysin

| Material | Mechanism of Erosion |
|---|---|
| Gelatin | Solubilization by water |
| Polyethylene glycol | Solubilization by water |
| Fatty acids and triglycerides | Solubilization by heat |
| Polyvinyl pyrrolidone | Solubilization by water |
| Starch | ″ |
| Cellulose ethers (eg., HPMC) | ″ |
| Hydrocolloidal gums and mucilages | ″ |
| (e.g., gum arabic, guar gum, gum | |
| tragacanth) | |
| Waxes (e.g., carnuba, bees) | Solubilization by heat |
| Polyacrylic acid derivatives and | Solubilization by water and pH |
| esters | |
| Shellac | Solubilization by pH |
| Cellulose acetate phthalate | ″ |
| Carboxy methylcellulose | Solubilization by water |

F. Erodible Plug

Second general release approach employs an erodible plug material placed above the dried drug solution within the pores. Such material can be dissolved or suspended in an oil or non-aqueous solvent and filled above the dried cytolysin solution, plugging the opening of the reservoir. Suitable materials are listed in Table 2.

G. Microfabricated Particles

The present invention provides microfabricated particles that are useful therapeutically in a variety of in vitro, in vivo and ex vivo applications, in particular, intravenous applications. The microfabricated particles have a selected nonspherical shape, uniform dimensions and contain a therapeutic agent in releasable form where the activity of the agent is expressed following its release from the device after binding to appropriate target cells within the bloodstream.

H. Representative Embodiments

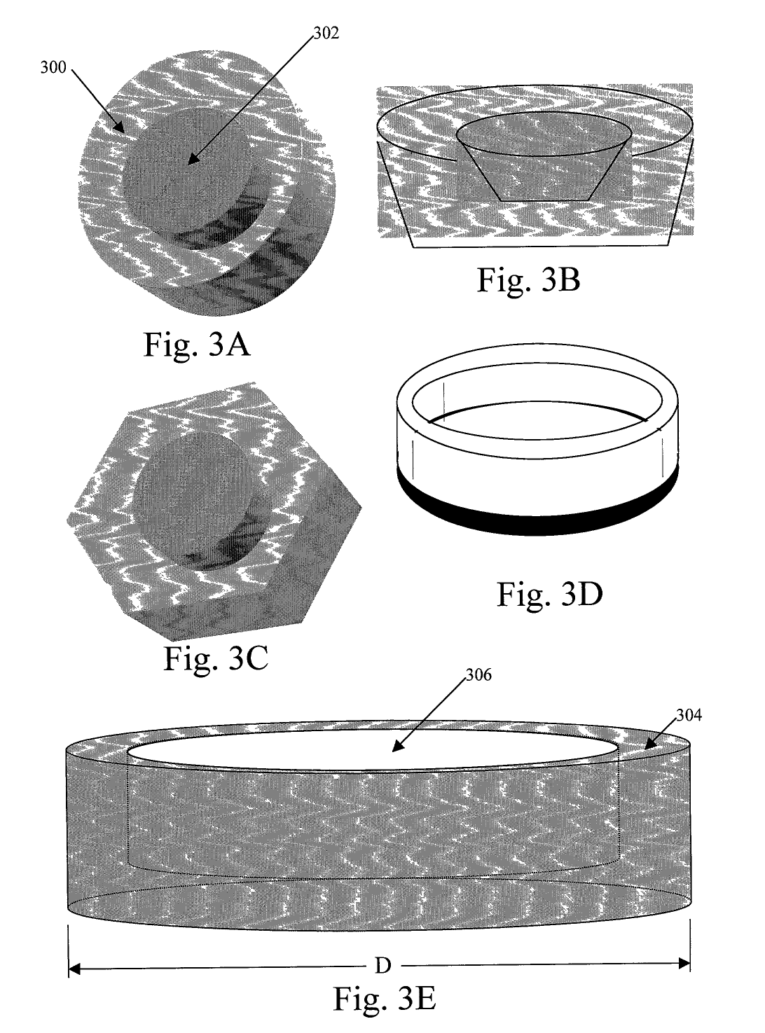

The shape, size, density, and composition of the microfabricated particle of the present invention are selected to favor the adhesive force (provided by the ligand grafted to face of the particle) as opposed to the forces which would tend to dislodge the particles once they have bound to the desired cell. The number and volume of reservoirs or pores in each particle is selected to provide adequate carrying capacity for the particular cytotoxic to be delivered. For example, devices designed to be used in typical applications are preferably substantially disk-shaped, cup-shaped, ring-shaped, or hexagonal-shaped. Exemplary embodiments of such disk-, cup-, ring- or hexagonal-shaped devices are illustrated in FIGS. 3A-1D. In reference to FIGS. 3A-3E, each particle is composed of substrate 300 and contains one or multiple pores or reservoirs 302.

FIG. 3E shows a disk-shaped particle composed of a thin disk material 104 with diameter D between about 0.5-10 microns and a thickness between about 0.5-10 μm. The disk is formed of a single polymer material which may contain the therapeutic agent (e.g., a cytotoxic drug) within pore or reservoir 306. Although non-erodible, biocompatible materials may be used, the preferred particles are formed of bioerodible materials, as described below.

The face of the particle containing the openings to the reservoirs or pores may be modified by the introduction of a 50-100 Å layer of reactive chemical groups. Typically these groups are added after formation of the particles. Methods of derivatizing a variety of glass, metal surface and polymer surfaces are well known. For example, amino or thiol groups can be grafted to the surface of polymers using glow discharge or “plasma” treatment.

Particles of the present invention are targeted by chemically linking appropriate ligands to the reactive groups on the face of the particle. Protein ligands are linked to amino- and thiol-reactive groups under conditions effective to form thioether or amide bonds respectively. The ligands illustrated are intended for binding the particle to selected target sites in or near a tumor. Methods for attaching antibody or other polymer binding agents to an inorganic or polymeric support are detailed, for example, in Taylor, R., Ed., Protein Immobilization Fundamentals and Applications, pp. 109110 (1991).

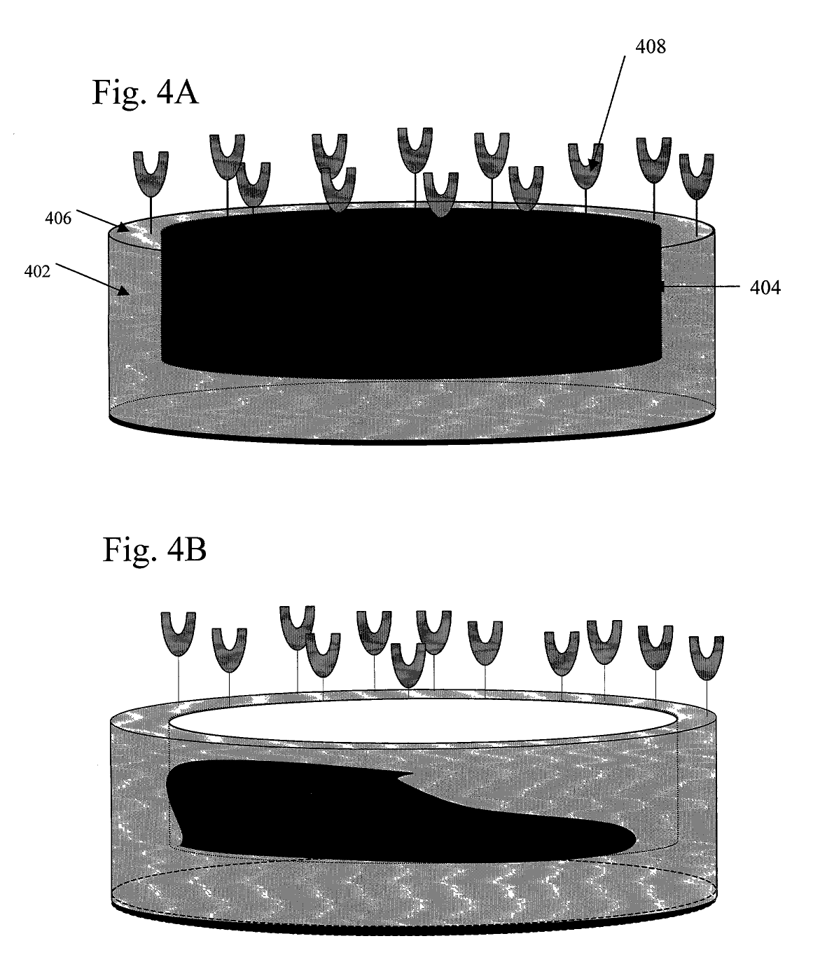

FIG. 4 shows a disk-shaped particle 402 having pores, 404, opening to the face of the particle, 406, and a layer of muco-adhesive ligands grafted to the face 408. In the embodiment shown in FIG. 4A, the pores are filled with aa aqueous mixture of a therapeutic agent and an excipient designed to delay the dissolution of the mixture for a few hours after the particle is injected. As shown in FIG. 4B, the solution filled in the reservoir is dried forming a drug/excipient plug which is designed to dissolve at a selected rate after injection. In other embodiments, the pore may contain an erodible plug to delay the release of the therapeutic agent.

The pores of the particle may be plugged with a material, such as a corrosion delay film. The corrosion delay layer is typically made of a material that gradually dissolves in the biochemical environment of the blood stream. Examples of such plug materials include thin layers of metals such as titanium, gold, silver, platinum, copper, and alloys and oxides thereof, gelatin, polysaccharides such as maltodextrins, enzyme-sensitive materials such as peptide polymers

The thickness of the corrosion delay layer may be selected to, for example, provide the desired delay of release within the blood stream, to allow the device to bind to its target before therapeutic agent is released. These layers may be applied by standard metal deposition procedures, sputtering, thin film deposition (see Wagner, J Oral Implantol 18(3):231-5;1992).

The optimal dimensions, shape and density of the substrate material of particles of the present invention depend on a striking a favorable balance between the dynamic movement of blood and the capacity of the particles to adhere to the endothelial cell layer of angiogenic blood vessels which supply blood to tumors. The maximum dimension of the devices (the diameter of the disk in the case of disk-shaped devices) is typically in the range between 0.5 and 10 microns.

The minimum dimensions of the particles are constrained only by the microfabrication process itself and the carrying capacity of each particle. As is described more fully below, it is recognized that “traditional” photolithography is limited to the microfabrication of structures greater than about 0.5 microns, but that substantially smaller structures (with dimensions contemplated in the present invention—e.g., 50-200 (nm diameter devices) may be produced using known X-ray and/or electron beam lithography methods.

Certain layers and coating, which may be contained in a device such as described above (e.g., a layer of ligands), can be as thin as a single layer of molecules. The minimum size again depends on the application. For example, in the case of devices made from biodegradable materials, the smaller the device, the faster it will dissolve. The stability of device of the present invention in a particular application may be readily determined by one of skill in the art using tagged (e.g., fluorescent or radiolabeled) devices in a model system.

Another important property of particles is the bioerodibility of the material employed in making the particle. Some metals, such as iron, are rapidly dissolved in aqueous media, whereas others, such as gold, are much more slowly eroded. Therefore, to achieve a desired rate of erosion, metals may be mixed in alloy.

A variety of bioerodible polymers, including polyglycolic, polylactic, polyurethane, celluloses, and derivatized celluloses may be selected, and a variety of charged polymers, such as heparin-like polysulfated or polycarboxylated polymers are suitable in forming one or more of the microstructure layers.

Further, the particles can be tagged so as to allow detection or visualization. For example, microdevices are rendered radioactive by implantation or surface attachment of radioactive isotopes such as I-123, I-125, I-131, In-111, Ga-67 and Tc-99m. Radioactive devices bound to particular regions of body can be identified by a radiation detectors such as the (-ray cameras currently used in scintigraphy (bone scans), resulting in identification and localization of such regions. Microdevices can also be tagged with fluorescent molecules or dyes, such that a concentration of microdevices can be detected visually.

The structural material used in forming the microstructure is selected to achieve desired erodibility and drug release properties. In the case of drug release, the structural material may be a one or more biodegradable polymer. Classes of biodegradable polymers include polyorthoesters, polyanhydrides, polyamides, polyalkylcyanoacrylates, polyphosphazenes, and polyesters. Exemplary biodegradable polymers are described, for example, in U.S. Pat. Nos. 4,933,185, 4,888,176, and 5,010,167. Specific examples of such biodegradable polymer materials include, for example, poly(lactic acid), polyglycolic acid, polycaprolactone, polyhydroxybutyrate, poly(N-palmitoyl-trans-4-hydroxy-L-proline ester) and poly(DTH carbonate).

I. Microfabrication Methods

The structural portion or substrate layer (i.e., microstructure) of the particles of the present invention may be microfabricated using any suitable microfabrication method, such as track-etching (PCTE) of polymer roll stock detailed in Example B, or the photolithography and photoablation methods detailed below. It will be appreciated that the particles can also be microfabricated using other microfabrication methods known to those skilled in the art, such as x-ray or electron beam lithography. Electron beam lithography has been used to produce sub-micron circuit paths (e.g., Ballantyne, et al., J. Vac. Sci. Technol. 10:1094 (1973)), and may be used (e.g., in combination with near field scanning microscopy) to generate and image patterns on the nanometer scale (see, e.g., Introduction to Microlithography, Thompson, et al., Eds., ACS Symposium Series, Washington D.C. (1983)).

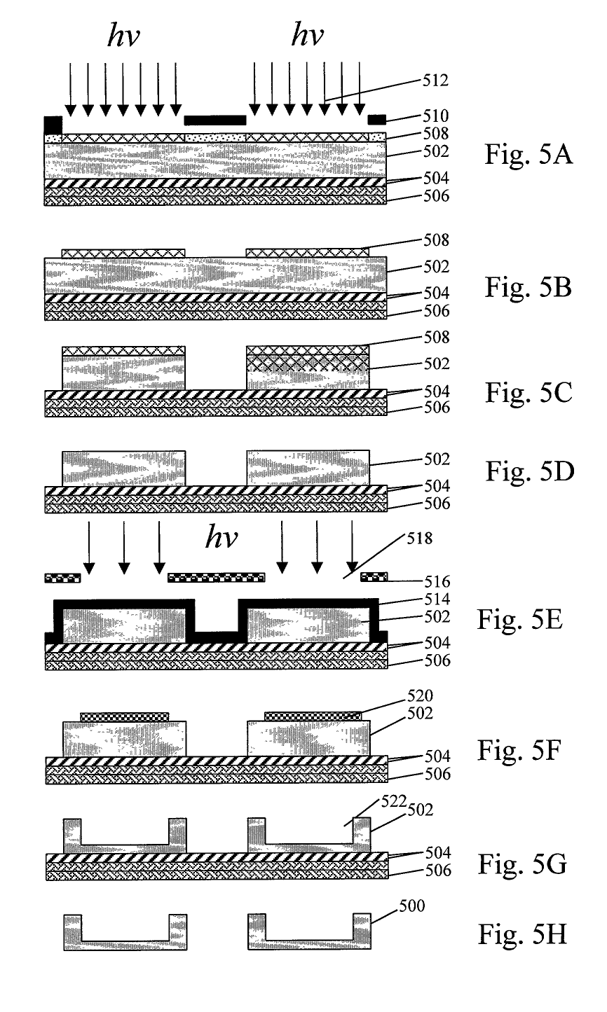

FIGS. 5A-5H illustrate the steps in forming a disk-shaped reservoir-containing particle 500 (FIG. 5E) by photolithographic techniques. As shown, the structure includes a polymer layer forming a planar expanse 502. This polymer expanse is formed according to conventional methods for deposition of metal layers, e.g., chemical vapor deposition, sputtering or the like, and/or methods for producing thin polymer sheet material.

As a first step in the process, the polymer layer is attached or otherwise bonded to a sacrificial layer 504, such as phosphorous doped silicon dioxide which is in turn coated onto a standard silicon wafer 506. The top of the polymer layer is coated with a photoresist layer 508 by chemical vapor deposition. Suitable negative- or positive-resist material are well known, e.g., Introduction to Microlithography, Thompson, et al., Eds, ACS Symposium Series, Washington D.C. (1983). Additional details on microfabrication methods useful in the manufacture of devices according to the present invention are described in, e.g., co-owned PCT patent publications WO 95/24261, WO 95/24472 and WO 95/124736.

The coated polymer layer is irradiated through a photomask 510 having a series of circular openings, such as opening 512, corresponding in size to the desired size of the particles. Methods for forming photomasks having desired photomask patterns are well known.

In the embodiment described with reference to FIGS. 5A-5D, the photoresist is a negative resist, meaning that exposure of the resist to a selected wavelength, e.g., UV, light produces a chemical change (indicated by cross hatching) that renders that altered resist resistant to etching by a suitable etchant. The appearance of the coated polymer layer after photomask irradiation UV FIG. 5C. As seen, the polymer layer 502 is now covered by a plurality of discrete disk-shaped resist elements, such as elements 508, corresponding in size to the planar dimensions of the desired particles.

The polymer layer is now treated with an etchant material effective to dissolve the polymer in the exposed areas of the polymer layer. In the case of a metal layer, the etchant may be a suitable acid solution; in the case of a laminate biodegradable polymer layer, the etchant could be an enzyme solution, an aqueous solution having a pH effective to break down the polymer, or an organic solvent known to dissolve the particular polymer. The polymer layer, after complete etching, has the appearance of FIG. 5C, which shows a series of disk-like, resist-coated elements on the sacrificial layer.

In the final preparation steps, the resist is removed by suitable chemical treatment (FIG. 5D).

FIGS. 5E-5H illustrate further photolithographic processing effective to produce disc-shaped particles containing pores or reservoirs, such as shown at 500. In this processing, the etched polymer/sacrificial layer structure or substrate shown in FIG. 5D are further coated with a positive resist material 514, as shown in FIG. 5E. The coated polymer is then irradiated through a second photomask 516 having a series of circular openings, such as opening 518, whose diameters correspond to the desired “internal” diameters of the reservoirs. The mask is aligned with the substrate, as shown, so that the mask openings are in registry with the already formed discs in the substrate.

Irradiation of the substrate through the photomask causes photo-induced changes in the resist (indicated by cross-dot pattern) that renders the irradiated regions susceptible to a selected etchant. The appearance of the coated laminate after photomask irradiation UV is shown in FIG. 5F. As seen, the polymer layer 502 is now covered by a plurality of discrete disk-shaped positive resist elements, such as elements 520, corresponding in size to the planar dimensions of the desired reservoirs. The polymer layer is now treated with a suitable second etchant material. The timing of the etching step is selected so that the layer is etched only partially creating blind pores in the layer. The appearance of the polymer after such etching is shown in FIG. 5G. As seen, this treatment has produced cylindrical pores, such as opening 530, in the center of each microstructure 500 in the substrate.

Removal of the sacrificial layer produces the free particles 500 shown in FIG. 5H. It will be appreciated that the particles formed as just described may be further treated by standard photolithographic techniques to produce other desired surface features and or layers. Further, reservoirs or pores may be filled with a material different from the microstructure material by known methods. For example, such reservoir may be filled with a selected therapeutic protein, such as interferon, insulin, various proteases, luteinizing releasing hormone and its analogs, and the like.

In another general approach, the particles are patterned from a substrate by excimer laser photoablation techniques. Methods of laser micromachining or dry etching have been described, e.g., U.S. Pat. Nos. 5,368,430, 4,994,639, 5,018,164, 4,478,677, 5,236,551, and 5,313,043. This method is most suited to a polymeric substrate, because of the ease with which a laser beam cans photoablate polymer structures.

Particles of the present invention may also be made by cutting or ‘punching’ individual particles from a variety of polymeric sheet-stock containing trak-etched pores. Such polymeric sheet-stock made of polycarbonate and polyester is commercially available. The pores are uniform, cylindrical, blind pockets or reservoirs on both faces. A non-porous backing material may be added to one face of the sheet, creating an asymmetric structure in which the pores open to only one face. Reactive chemical groups such as amino functions may be introduced onto the face of the sheet to which the pores open.

J. Microstructure Surface Structures

The term “molecular coating” is used herein to describe a coating, which is bound to one surface (face) of a particle. The molecular coating is bound directly to the surface of the particle or grafted to the surface via a chemical bond to an electron donating group, e.g. —NH₂, OH or the like derivatized onto or associated with the surface of a structural layer of the particle. In a preferred embodiment, the molecular coating is limited to the face of the particle to which the reservoirs or pores empty. Molecular coatings that confer the ability for the particle to bind to the mucin layer covering the small and large intestine (muco-adhesive ligands) are preferred.

FIG. 5C illustrates a general embodiment of a particle containing a grafted layer of reactive ligands 512. The particle contains pores or reservoirs 514 each of which is filled with a mixture of cytotoxic drug and an excipient (or blend of excipients) which are selected to delay dissolution of the mixture (indicated by the stippled pattern within the pores). In one general embodiment, the ligand is a growth factor such as FGF useful for binding the particle to surface of proliferating endothelial cells. As illustrated in FIG. 5D, the cytotoxic drug solution is dried after filling into the reservoirs (as indicated by the retracted stippled pattern within each reservoir.

To facilitate tracking of a therapeutic particle of the present invention, one of the structural or coating elements of the particle may be designed to be detectable using, for example, X-radiation, scintigraphy, nuclear magnetic resonance, optical inspection (e.g., color, fluorescence), or ultrasound.

K. Therapeutic Agents

Particles of the present invention consist of microfabricated structural elements (particles) encapsulating a therapeutic agent within an internal reservoir and coating (such as ligands). The therapeutic agent may be filled into the pores or reservoirs during or after microfabrication of the particle.

The activity of the therapeutic agent is expressed by exposure of the particle to the aqueous environment of the blood stream. The target site can be either the proliferating endothelium forming blood vessels which supply blood to tumors or the tumor cells themselves. The therapeutic agent contained in the therapeutic particles of the present invention is releasable. A releasable agent is a therapeutic compound, such as a drug, that is designed to be released from the reservoirs of the particle while the particle is bound to the desired target cell

L. Particle Suspension

The invention includes a suspension of particles of the type described above for use in administering a therapeutic agent via the IV route. To form the suspension, particles as described above are suspended in any suitable aqueous carrier vehicle. A suitable pharmaceutical carrier is one that is non-toxic to the recipient at the dosages and concentrations employed and is compatible with other ingredients in the formulation.

### II. APPLICATIONS

Particles of the present invention can be administered to a subject in need of therapeutic intervention via the IV route.

As discussed above, particles of the present invention are particularly useful in the delivery of cytotoxic drugs to tumors.

### III. EXAMPLES

Microfabricated Particles for Delivery of Melittin to the Angiogenic Blood Vessels Supplying Tumors

FIGS. 5A-5H illustrate the steps in forming a disk-shaped particle by photolithographic techniques on a standard 4″ type single crystal (SC) silicon wafer. 100 nm of silicon oxide is thermally grown on the SC silicon substrate at 1000° C. under “wet” conditions to form an etch-stop layer (not shown). A sacrificial layer of poly-crystalline silicon (poly; 1830 nm) is deposited on the etch-stop layer by low pressure chemical vapor deposition (LP-CVD) in a Tylan furnace (605° C., 300 mTorr, 100.0 sccm SiH₄) and the wafer is annealed for 1 hour at 1000° C. to remove residual stresses. A 900 nm layer of LTO is deposited on the sacrificial poly by LP-CVD in a Tylan furnace (450° C., 300 mTorr, 60.0 sccm SiH₄, 90.0 sccm O₂, 0.4 sccm PH₃) to form the microparticle layer, and again the wafer is annealed for 1 hour at 1000° C. to densify the LTO. The wafers are patterned on the LTO surface by UV photolithography GCA 6200 DSW Wafer Stepper (GCA MANN Products) to yield a photo-resist (PR) pattern of circular-shaped areas about 100-200 microns on diameter. The wafer is then baked. The exposed areas of the LTO on the PR patterned LTO surface are etched in a LAM plasma etcher (850W @ 0.38 cm gap, 2.8 Torr, 120.0 sccm He, 30.0 sccm CHF₃, 90.0 sccm CF₄). Remaining photoresist is removed in pirhana (5 parts 18M H₂SO₄, 1 part 30% H₂O₂) to yield a wafer having separate microparticles attached to an underlying poly layer.

The remaining LTO particles are coated with a second, positive, resist layer, exposed to UV light for a second time through a photomask with a finer pattern of circular openings. The diameter of the opening and the density of the opening within the photomask are selected to provide suitable pores or reservoirs of 0.5-5 microns in diameter in the LTO particles. The exposed layer is then treated with a second etchant material effective to partially dissolve the polymer in the exposed areas creating a plurality of cylindrical- or cone-shaped pores or reservoirs in each particle. Importantly, conditions are adjusted so the sheet is etched to a desired depth, but not completely through the polymer layer. In the case of a metal layer, the etchant may be a suitable acid solution; in the case of a biodegradable or biocompatible polymer layer, the etchant could be an enzyme solution, an aqueous solution having a pH effective to break down the polymer, or an organic solvent known to dissolve the particular polymer.

Next, the upper surface of the particles is chemically modified to produce reactive chemical groups such as primary amino or thiol, groups. A preferred method of introducing such groups into the first few molecular layers of silicon uses treatment with the silane reagents described below. For polymer material, the gas plasma treatment described below is preferred.

The sacrificial poly layer is then removed by a wet etch in 6M KOH at 80° C. (1-2 minutes) to release the particles into solution. After the particles are released the pH is promptly reduced to below 8 and the particles are stored in neutral H2O (resistivity>17.8 Mohms/cm).

The particles are suspended in PBS and ligands are grafted to the particle face via these reactive chemical groups using the methods described below.

The melittin solution is filled into the pores at this point in the process. The particles are thoroughly washed in distilled water, collected on a filter and dried under reduced pressure. The particles are resuspended in a degassed solution of melittin plus excipients as described below. The suspension is subjected to reduced pressure to insure that trapped air is forced from the pores in the particles. The are fully immersed in the solution and the pressure is elevated slightly above atmospheric to insure that the solution enters all the pores. The particles are once again trapped on a filter and dried using one of the three methods described below.

A. Grafting of Primary Amine Groups to the Face of Particles

1. Silicon Glass Surfaces

Reactive primary amino-groups are introduced on the silicon glass surfaces using 3-aminopropyltriethyloxysilane or N-(2-Aminoethyl)-3-aminopropyltrimethyloxysilane (Pierce Chemical Co., Rockford, Ill.). The top surface of the particles (still attached to the sacrificial layer) is washed in dilute HCl. The selected silane reagent is dissolved in anhydrous acetone (20 μl/mL) and applied to the particle array for 6 hours at 60° C.

2. Polymer Surfaces

A Glow Discharge or Gas Plasma technique is used to introduce reactive primary amino groups into the face of the polymer sheets. Gas Plasma Surface Modification is done in a vacuum chamber in the presence of ammonia vapor and has been used to modify plastics and other polymer surfaces (Kany et al, Biomaterials 18(16):1099-107;1997 and Siphia, Biomater Artif Cells Artif Organs 18(3)37-46;1990 and Benedict and Williams, Biomater Med Devices Artif Oragns 7(4):477-93;1979 and Liu, et al, J Biomed Mater Sci 27(7):909-15;1993. Equipment for conducting such processing is available on a contract basis at MetroLine, Inc. (251 Corporate Terrace Corona, Calif. 91719).

Gas plasma is ionized gas, the fourth state of matter. A plasma is formed when a gas, in this case ammonia, is exposed to energy, generally an electric field. Cold gas plasma reactions are conducted in a vacuum chamber, built of either Pyrex, quartz or aluminum, and having either an internal or an external electrode configuration. Low-pressure gases are then ionized using a radio frequency (RF) power, at 13.56 MHz. The RF energy strips electrons from the gas species, producing free electrons, ions and excited molecules. As the active molecules recombine with the electrons, photons are released, causing the “glow” which is associated with gas plasmas. Each gas type “glows” with a specific color. As soon as the RF power is turned off, the gas molecules recombine to form stable molecules, and are evacuated from the chamber.

Gas plasma surface modifications used here falls into the categories of molecular modifications (often referred to as ‘etching’ or molecular modification of a surface) will result in a new chemical surface without actually depositing any additional materials.

There are a number of critical parameters, which are controlled during the plasma treatment cycle. Any change in these parameters will influence the outcome of the modification. They are as follows:

Gas Type Power

Pressure

Flow

Exposure Time

Chamber and Fixture Configuration

Various other factors may effect treatment, such as ambient conditions, relative humidity during component molding, surface contamination of the substrates, or polymer lot-to-lot variations. A molecular modification alters the chemical structure of the surface of an organic material, in this case polycarbonate. Ammonia gas also ionizes under the influence of the electrical discharge. Molecules traveling at high speeds during the ionization cycle impact with the surface of the polycarbonate causing the polycarbonate polymer backbone to fracture and form reactive species such as radicals. Some of the ionized ammonia molecules then attach themselves to the substrate surface, thus forming a layer of covalently bound primary amino groups.

Ammonia plasma discharge modification generally involves from 25 to 250 angstroms of the substrate surface and thus does not alter the bulk properties of the underlying polymer substrate.

Reactive amine groups can also be introduced into polymer surfaces using glow discharge techniques in the presence of alkylamine vapors such as butylamine (Tseng and Edelman, J Biomed Mater Res 42(2):188-98;1998) and ethylene-diamine (Denizli et al, J Biomater Sci Polym Ed 10(3):305-18;1999.

Radiofrequency glow discharge treatment in the presence of water or H₂O₂ vapor, or glow discharge in air (O₂) may also be used to introduce reactive hydroxyl groups into polymer surfaces (Patterson, et al, ASAIO 41(3):M625-9;1995 and Kang et al, Biomaterials 17(8):841-7;1996 and Vargo et al, J Biomed Mater Res 29(6):767-78;1995 and Ozden et al, Dent Mater 13(3):174-8;1997). Water-soluble condensing agents such as carbodiimide are used to link amino-containing protein ligands to the —OH-modified polymer surface. Polycarbonate can be modified by introduction of reactive double bonds by treatment with glycidyl acrylate (Karmath and Park, J Appl Biomater 5(2):163-73;1994).

It should be noted that other surface modification techniques such as graft polymerization by h-irradiation may be used to introduce reactive groups to the face of the particles (see for example, Ikadal, Biomaterials 15(10):725-36;1994 and Amiji and Park, J Biomater Sci Polym Ed 4(3):217-34;1993 and Kamath and Park, J Appl Biomater 5(2):163-73;1994).

B. Creation of Microparticle Suspensions

The array of silicon or polymer particles with 0.5-5 μm diameter pores or reservoirs and surface reactive amino groups is further processed to yield a suspension of individual particles in the 5-10 μm range. In the case of silicon particle arrays, the sacrificial layer is removed using standard techniques to produce a suspension of silicon microparticles. In the case of polymer sheets, the individual micropartciles may be punched out of the polymer sheet using a micropunch apparatus. Alternatively, individual microparticles may be cut from the polymer sheet using chemical or enzymatic etchants or laser knives.

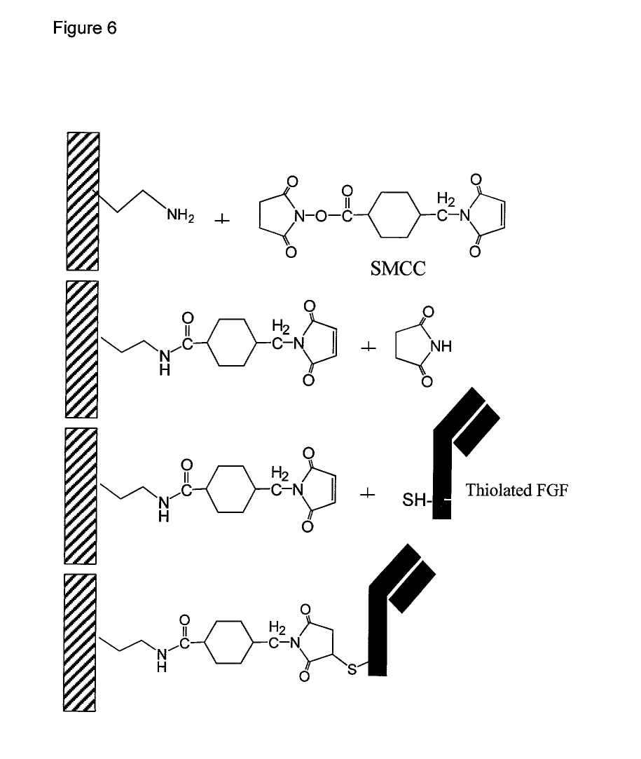

C. Chemical Coupling of Ligands to Surfaces of Amino-Modified Microparticle Suspension (FIG. 6)

In each case, the particle suspension is submerged in a solution of SMCC or similar hetero-bifunctional reagent (Pierce Chemical Company, Rockford, Ill. 61105), introducing thiol-reactive maleimide groups onto the face of the particle. The reaction is virtually stoichiometric (FIG. 6). Heterobifunctional reagents with extended spacer arms also be used to improve coupling efficiencies by reducing steric hindrance (Bieniarz et al, Bioconjugate Chem 7:88-95; 1996). As the particle array is removed from the SMCC solution, some solution may remain in the pores. The particle array is then placed into vacuum chamber. When the vacuum is applied, water vapor moves out of the chamber and is condensed. Pressure within vacuum chamber may be alternately reduced and then raised to insure that any trapped air is cleared from the pores. Within the vacuum chamber, the particle array is rinsed by spaying the sheet from nozzle with water, which is collected in drainage area and removed by drain. The particle sheet next advances into vacuum chamber. A high vacuum is applied and water remaining within the pores evaporates and water vapor passes out of the chamber. The pores are now dry. The silicon or polymer sheet containing the thiol-reactive maleimide groups is now ready for the ligand modification step.

The silicon or polymer sheet with 0.5-10 μm diameter pores or reservoirs and thiol-reactive maleimide surface groups introduced above is submerged in a solution highly purified FGF solution. FGF is obtained as a lyophilized powder from Selective Genetics, Inc (San Diego, Calif.). A solution of twenty milligrams of FGF is made in 1 mL of phosphate-buffered isotonic saline (PBS). FGF used for this purpose contains an unpaired reactive thiol group (an unpaired cysteine residue at amino acid position 78). Ligands without reactive thiol groups may be modified by thiolation using SPDP following the procedure of Carlsson et al (Biochem. J 173:723-37;1978). In such a case, conditions are adjusted to yield 1.5-6 —SH groups per ligand molecule after mild reduction. The thiolated ligand is chemically linked to the thiol-reactive maleimide groups (FIG. 6). The time and temperature are adjusted to insure adequate coupling of the thiol-containing ligand in to the thio-reactive polymer. As the polymer sheet is removed from the ligand solution, some solution may remains in the pores. The polymer is then washed either by placement in distilled water (not shown) or sprayed with distilled water. In the case of spray washing, the sheet passes into vacuum chamber. When the vacuum is applied, water vapor moves out of the chamber and is condensed. Within the vacuum chamber, the sheet is rinsed by spaying the sheet from nozzle with water, which is collected in drainage area and removed by drain. After washing, the sheet next advances into vacuum chamber. Within this chamber, the film is gently dried to insure that the pores are emptied of any fluid. In the case of freeze drying, a flat heat exchanger is placed in good thermal contact (directly below) the polycarbonate film. Liquid refrigerant at temperatures ranging from −20° C. to −60° C. (such as Freon or a cold liquid such as liquid nitrogen) is passed through the heat exchanger in order to freeze any water remaining on the film or within the pores. The pressure is reduced until all the water sublimes. In this example, drying is achieved by evaporation of the remaining water under reduced pressure in vacuum chamber, or by passage of a stream of warm air or an inert gas such as nitrogen over the surface of the film, or by freeze drying as mentioned above. In the case of vacuum drying exemplified here, a high vacuum is applied and water remaining within the pores evaporates and water vapor passes out of the chamber. The pores are now dry. The sheet containing the lectin chemically grafted to the surface advances to the filling step.

D. Filling Reservoirs with Cytotoxic Drug Solution

1. Mixing Cytotoxic Drug with Excipients which Provide Delayed Release from Micro-reservoirs

A solution of 50 mg/mL melittin (Sigma Chemical Company) is made in PBS. A range of water-soluble excipients can be added to this solution to delay dissolution when dried. These include polymers, dextrans, maltodextrins, gelatins, disintegrants such as Explotab, polyplasdone, amberlite IRP 88, maize or potato starch and Elcema P100.

2. Filling Reservoirs with Cytotoxic Drug/excipient Solution

The silicon or polymer microparticle suspension with pores and chemically grafted FGF groups introduced as detailed above is submerged in a degassed solution of melittin/excipients in a sealed chamber. The suspension is subjected to reduced pressure to insure that trapped air is forced from the pores in the particles. The are fully immersed in the solution and the pressure is elevated slightly above atmospheric to insure that the solution enters all the pores. The particles are trapped on a filter and dried using one of the three methods described below.

To remove any trapped air within the reservoirs in the submerged microparticles, the pressure within the chamber is reduced, and then raised slightly above atmospheric pressure.

3. Drying

After filling of melittin/excipient solution into the reservoirs of the FGF-modified silicon or polymer micropartciles, drying is achieved by one (or a combination) of three methods. Water is removed by evaporation under reduced pressure in a vacuum chamber, or by passage of a stream of warm air or an inert gas such as nitrogen over the surface particles collected on a filter, or by freeze frying. In the case of freeze drying, a flat heat exchanger is placed in good thermal contact (directly below) the filter on which the microparticle suspension has been collected. Refrigerant fluid at temperatures ranging from −20° C. to −60° C. (such as Freon or a cold liquid such as liquid nitrogen) is passed through the heat exchanger flowing into port and passing out port in order to freeze any water remaining within the pores. The pressure is reduced until all the water sublimes.

What is claimed is:

1. Asymmetric microparticles for intravenous administration in treating tumors, having:
(i.) uniform sizes, e.g., in the range 0.5 to 10 μm,
(ii.) at least one internal reservoir which communicates through at least one pore with the front face of said particle,
(iii.) each reservoir containing a releasable cytotoxic agent,
(iv.) said pore and/or reservoir filled or covered with release-delaying material, and
(v.) a layer of ligand molecules chemically grafted to the same face of the particle as the pore openings.

2. A particle of claim 1, wherein the release-delaying material delays release of the cytotoxic agent for 1-48 hours after injection.

3. A particle of claim 1, wherein said reservoir or said pore is covered with a semipermeable membrane.

4. A particle of claim 1, wherein a coating of a hydrophilic polymer, such as polyethylene glycol, effective to extend the circulation lifetime of the particles in the bloodstream, is chemically grafted to all faces of the particle surface.

5. A particle of claim 4, wherein the ligand is coupled to a spacer arm sufficient to extend said ligand beyond the hydrophilic polymer layer.

6. A particle of claim 1, wherein following release from said reservoir, the cytotoxic agent enters the surface membrane of juxtaposed cells and causes cytolysis.

7. A cytotoxic agent of claim 6, wherein the cytolytic agent is bee venom melittin.

8. A cytotoxic agent of claim 6, wherein the cytolytic agent is paradaxin, hemolysin, amoebapore, pilosulin, magainin, lentivirus lytic peptide, NK-lysin or perforin.

9. A particle of claim 1, wherein the shape is disc-like or hexagonal-like.

10. A particle of claim 1, wherein the front face is grafted with a layer of reactive amino or thiol groups by plasma (glow) discharge or by sialylation methods.

11. A particle of claim 10, wherein the layer of reactive amino or thiol groups is used to chemically link ligands to the front face of the particle.

12. A particle of claim 1 wherein the ligand binds to receptors overexpressed on tumor cells or angiogenic vascular endothelial cells.

13. A particle of claim 12, wherein the ligand is FGFb.

14. A particle of claim 12, wherein the ligand is selected from VEGF, c-erbB-2 ligand, RGD-type tumor targeting cyclic peptides or folate.

15. A particle of claim 1, wherein the ligand is an antibody or antibody fragment which binds to receptors overexpressed on tumor cells or angiogenic vascular endothelial cells.

16. A ligand of claim 15, wherein the antibody or fragment thereof binds to growth factor receptors overexpressed on tumor cells or angiogenic vascular endothelial cells.

17. A antibody or fragment thereof of claim 15, wherein the growth factor receptor is FGFr, VEGFr or c-erbB-2 receptor.

18. A ligand of claim 15, wherein the antibody of fragment thereof binds to integrin receptors overexpressed on tumor cells or angiogenic vascular endothelial cells.

19. A antibody or fragment thereof of claim 18, wherein the integrin receptor is e-selectin, p-selectin or v3.

20. A particle of claim 1, wherein said particles are formed of a biodegradable polymer material.

21. A particle of claim 1, wherein the particles contain a radioactive material.

22. A particle of claim 1, wherein the release-delaying material is co-mixed with the cytotoxic agent held within the reservoir.

23. A particle of claim 1, wherein the release-delaying material is layered above the cytotoxic agent within said reservoir.

24. A particle of claim 1, wherein the release-delaying material forms a plug within or covering said pore.

25. A particle of claim 1, wherein the release-delaying material is a semipermeable membrane covering said pore.

26. A particle of claim 1, wherein the release-delaying material consists of gelatin, polyethylene glycol, fatty acids or esters, polyvinyl pyrrolidone, starch, dextrans or maltodextrins, hydrocolloidal gums or mucilages, waxes, polyacrylic acids, shellac, cellulose acetate phthalate or carboxymethylcellulose.

27. A microfabrication method for producing asymmetrical particles for use in claim 1, comprising exposing a sheet of particle-forming material to a photoablating light source through a series of photomasks forming a reticular lattice pattern on said sheet corresponding to the desired particle external size, shape and interior volume and continuing said exposure until the desired particles are formed.

28. A method for treating patients with solid tumors, wherein asymmetric microparticles, having:
(vi.) uniform sizes, e.g., in the range 0.5 to 10 μm,
(vii.) at least one internal reservoir which communicates through at least one pore with the front face of said particle,
(viii.) said internal reservoir containing a releasable cytotoxic agent,
(ix.) said pore and/or reservoir filled or covered with release-delaying material, and
(x.) a layer of ligand molecules chemically grafted to the same face of the particle as the pore openings
are injected intravenously into the patient.

29. A method of claim 28, wherein following release from said reservoir, the cytotoxic agent enters the surface membrane of juxtaposed cells and causes cytolysis.

30. A method of claim 29, wherein the cytolytic agent is bee venom melittin.

31. A method of claim 29, wherein the cytolytic agent is paradaxin, hemolysin, amoebapore, pilosulin, magainin, lentivirus lytic peptide, NK-lysin or perforin.

32. A method of claim 28, wherein the ligand binds to receptors overexpressed on tumor cells or angiogenic vascular endothelial cells.

33. A method of claim 32, wherein the ligand is selected from VEGF, c-erbB-2 ligand, RGD-type tumor targeting cyclic peptides or folate.

34. A method of claim 32, wherein the ligand is an antibody or antibody fragment which binds to receptors overexpressed on tumor cells or angiogenic vascular endothelial cells.

35. A method of claim 34, wherein the antibody or fragment thereof binds to growth factor receptors overexpressed on tumor cells or angiogenic vascular endothelial cells.

36. A method of claim 35, wherein the growth factor receptor is FGFr, VEGFr or c-erbB-2 receptor.

37. A method of claim 34, wherein the antibody of fragment thereof binds to integrin receptors overexpressed on tumor cells or angiogenic vascular endothelial cells.

38. A method of claim 37, wherein the integrin receptor is e-selectin, p-selectin or v3.

39. A method of claim 28, wherein said particles are formed of a biodegradable polymer material.

40. A method of claim 28, wherein the release-delaying material is co-mixed with the cytotoxic agent held within the reservoir.

41. A method of claim 28, wherein the release-delaying material is layered above the cytotoxic agent within said reservoir.

42. A method of claim 28, wherein the release-delaying material forms a plug within or covering said pore.

43. A particle of claim 28, wherein the re material is a semipermeable membrane covering said pore.

44. A method of claim 28, wherein the erodable material consists of gelatin, polyethylene glycol, fatty acids or esters, polyvinyl pyrrolidone, starch, dextrans or maltodextrins, hydrocolloidal gums or mucilages, waxes, polyacrylic acids, shellac, cellulose acetate phthalate or carboxymethylcellulose.
